(19) United States
(12) Patent Application Publication
(10) Pub. No.: US 2024/0341598 A1
(43) Pub. Date: Oct. 17, 2024

(54) Wireless Intraocular Pressure Sensor

(71) Applicant: Queen's University at Kingston, Kingston (CA)

(72) Inventor: Yong Jun Lai, Kingston (CA)

(21) Appl. No.: 18/633,882

(22) Filed: Apr. 12, 2024

Related U.S. Application Data
(60) Provisional application No. 63/459,161, filed on Apr. 13, 2023.

(51) Int. Cl. A61B 3/16

(52) U.S. Cl. CPC A61B 3/16

(57) ABSTRACT

Apparatus and method for measuring and monitoring intraocular pressure (IOP) of a subject includes a contact lens disposed on the subject's eye, the contact lens having a polymer matrix and a nanowire network embedded in the polymer matrix. An electrically conductive coil is disposed in close proximity to the contact lens such that it electromagnetically couples with the nanowire network of the contact lens. A resonant frequency of the electrically conductive coil is measured and correlated with the IOP of the subject. Changes in IOP result in deformation of the nanowire network of the contact lens and a resulting change in the resonant frequency, which is measured and correlated with the change in IOP. The electrically conductive coil is adapted to be worn by the subject and may be mounted on a pair of glasses.

### RELATED APPLICATION

This application claims the benefit of the filing date of Application No. 63/459,161 filed on Apr. 13, 2023, the contents of which are incorporated herein by reference in their entirety.

### FIELD

The invention relates generally to the field of intraocular pressure measuring and monitoring. More specifically, the invention relates to a contact lens that may be comfortably worn by a subject to provide long term measurements and monitoring of the subject's intraocular pressure.

### BACKGROUND

Glaucoma is a serious medical condition that can cause blindness and affects more than 400,000 Canadians [1]. The disease is commonly caused by elevated intraocular pressure (IOP). The threshold for glaucoma diagnosis is an IOP of over 21 mmHg [2]. Over time, this causes damage to the optic nerve can lead to vision loss. Glaucoma is difficult to diagnose, since IOP varies throughout the day due to both a person's physical state and environmental factors [3]. The current standard diagnosis procedure, called tonometry, involves a one-time measurement of eye pressure, typically at an appointment with a physician. This does not give a full overview of the patient's average IOP and can lead to an inaccurate or late diagnosis [2], [3].

To solve this issue, continuous IOP monitoring systems in the form of “smart” contact lenses have been proposed. Such contact lenses contain very small circuits that act as strain gauges and can measure the expansion of the eye which is directly correlated with IOP [2], [4]. Existing solutions all face drawbacks such as: the lenses employ circuitry that is difficult to manufacture (e.g., liquid wires within the lens), the lens requires electrode connections for measurement, or the lens body is up to five times thicker than a standard contact lens, potentially causing discomfort for the wearer [2], [5].

### SUMMARY

One aspect of the invention relates to a contact lens comprising a polymer matrix; and a nanowire network embedded in the polymer matrix.

Another aspect of the invention relates to apparatus for measuring intraocular pressure (IOP), comprising: a contact lens as described herein; and an electrically conductive coil adapted to share radiofrequency (RF) energy with the nanowire network of the contact lens.

According to embodiments, the electrically conductive coil has a selected resonant frequency.

According to embodiments, the resonant frequency changes in response to a change in configuration of the nanowire network.

According to embodiments, the change in resonant frequency is correlated with a change in IOP.

The apparatus may further comprise a device connected to the electrically conductive coil that produces and measures the frequency of the RF energy.

In various embodiments, the electrically conductive coil may be connected to a control unit; wherein the control unit comprises one or more of signal generator, oscillator, amplifier, analogue to digital converter (ADC), logic circuitry, processor, memory device, wireless communications device, power supply, and actuator.

The control unit may provide one or more of data acquisition, processing, identification of resonant frequency, and wireless data communications to a connected device.

The electrically conductive coil and the control unit are adapted to be worn by a user.

In one embodiment electrically conductive coil and the control unit are adapted to be mounted on a pair of glasses.

In various embodiments the polymer matrix comprises at least one of a hydrogel and polydimethylsiloxane.

In one embodiment the polymer matrix comprises poly (2-hydroxyethyl methacrylate).

Another aspect of the invention relates to a method for measuring and/or monitoring IOP of a subject, comprising: disposing a contact lens on the subject's eye, the contact lens comprising a polymer matrix and a nanowire network embedded in the polymer matrix; disposing an electrically conductive coil in close proximity to the contact lens, wherein the electrically conductive coil electromagnetically couples with the nanowire network of the contact lens; measuring a resonant frequency of the electrically conductive coil; and correlating the resonant frequency with the IOP of the subject.

The method may comprise measuring the resonant frequency of the electrically conductive coil at two or more instances in time; and correlating a change in resonant frequency over the two or more instances in time with a change in IOP of the subject.

The method may comprise connecting the electrically conductive coil to a control unit; wherein the control unit comprises one or more of signal generator, oscillator, amplifier, analogue to digital converter (ADC), logic circuitry, processor, memory device, wireless communications device, power supply, and actuator.

According to various embodiments of the method, the control unit may provide one or more of data acquisition, processing, identification of resonant frequency, and wireless data communications to a connected device.

According to various embodiments of the method, the electrically conductive coil and the control unit may be adapted to be worn by a user.

According to various embodiments of the method, the electrically conductive coil and the control unit may be adapted to be mounted on a pair of glasses.

According to various embodiments of the method, the polymer matrix may comprise at least one of a hydrogel and polydimethylsiloxane.

According to various embodiments of the method, the polymer matrix may comprise poly (2-hydroxyethyl methacrylate).

### BRIEF DESCRIPTION OF THE DRAWINGS

For a greater understanding of the invention, and to show more clearly how it may be carried into effect, embodiments will be described, by way of example, with reference to the accompanying drawings, wherein:

FIGS. 1A-1D are diagrams of different configurations of contact lenses with silver nanowires according to various embodiments of the invention.

FIGS. 2A-2C are photomicrographs showing disbursement of nanowires in a lens at three different nanowire concentrations.

FIG. 3 is a diagram of a coil holder used to make an inductive reading coil, according to one embodiment.

FIG. 4 is a photograph of an inductive reading coil, according to one embodiment.

FIG. 5 is a block diagram of an experimental set-up used to test nanowire contact lenses, according to one embodiment.

FIG. 6 is a plot showing resonant frequency of a reading coil alone (Without Lens) and with a nanowire lens (With Lens) introduced at a distance of 0.7 cm from the reading coil, according to one embodiment.

FIG. 7 is a plot showing change in resonant frequency of a reading coil magnetically coupled to a nanowire lens during manual deformation, according to one embodiment.

FIG. 8 is a diagram of an implementation of a reading coil and control unit, according to one embodiment.

FIG. 9 is a schematic diagram of an implementation of a reading coil, control unit, and nanowire lens, according to one embodiment.

### DESCRIPTION OF EMBODIMENTS

Described herein is a wireless IOP measurement contact lens with an easily manufacturable circuit using nanowires and a thin lens structure that can be reliably fabricated. The lens may be made from a polymer such as, such as, for example, polydimethylsiloxane (PDMS) or a hydrogel such as poly (2-hydroxyethyl methacrylate) (pHEMA), or a combination thereof. The nanowires may be made from various materials, including, but not limited to, silicon, germanium, carbon, and various conductive metals, such as, but not limited to, silver, gold, and copper, and combinations thereof. A contact lens and a measurement system according to embodiments offer advantages over prior systems, for example, they are able to be worn comfortably by a human for up to 24 hours to enable long-term measurement of IOP, embedded features (i.e., nanowires) in the lens do not impair the vision of the wearer such that it would prevent them from performing day-to-day tasks, and apparatus used to interface with the contact lens can be placed upon the wearer's body without causing any physical impairment. In addition, the measuring system is based on electromagnetic or inductive coupling between the nanowires of the lens and an electrically conductive reading coil placed in close proximity to the lens. Hence there is no direct (wired) electrical connection to the lens, and the system is referred to herein as “wireless”.

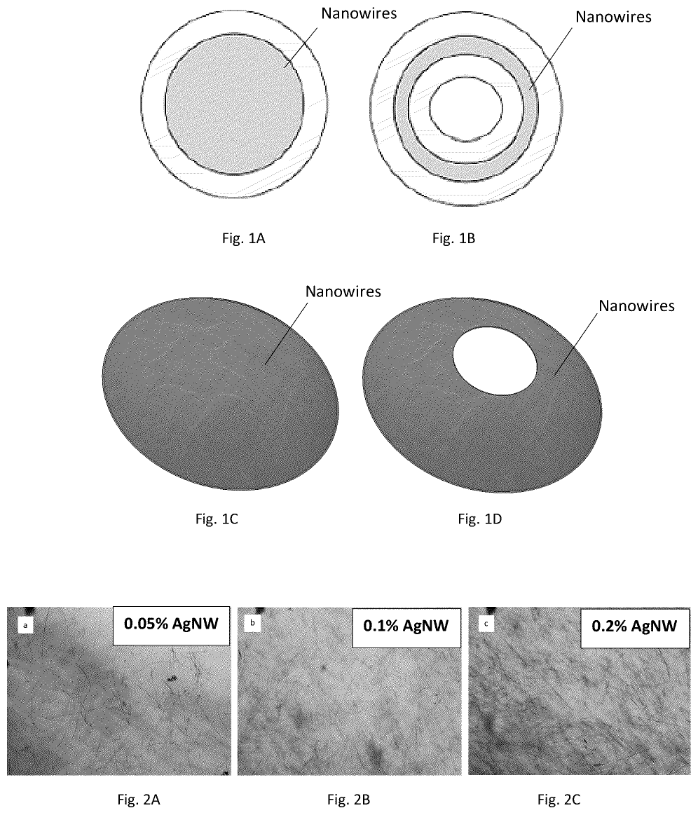

According to embodiments, a lens may comprise a layered structure having two or more layers of polymer material, with nanowires embedded in at least one layer and/or “sandwiched” between layers. In other embodiments a lens may comprise a single layer of polymer material with embedded nanowires. Embedded nanowires may be prepared by mixing nanowires with liquid polymer material and then curing to form a lens or a layer for a lens. In some embodiments the lens may have a maximum center thickness of 300 μm. In some embodiments the lens may have a maximum center thickness of 250 μm. Examples of various configurations are shown in FIGS. 1A-1D, and include a full (closed) lens with a circular area of nanowires in the centre (FIG. 1A), a ring-shaped (open) lens with a ring of nanowires (FIG. 1B), a full lens substantially completely embedded with nanowires (FIG. 1C), and a ring-shaped (open) lens substantially completely embedded with nanowires (FIG. 1D). In some embodiments the hole of the ring-shaped lens may be about, e.g., 3-6 mm in diameter, substantially in the center. Other configurations such as those have more than one hole in the lens may also be provided.

To demonstrate certain aspects of the invention, circular closed and open (i.e., ring-shaped) contact lenses with embedded silver nanowire (AgNW) mesh networks or dispersed AgNW throughout the contact lenses were made to act as strain gauges. A portable vector network analyzer was connected to an inductive reading coil placed in close proximity to the lenses to measure frequency response data corresponding to changes in shape (e.g, flexing) of the contact lenses, as a simulation of eye expansion, when wirelessly coupled with the AgNW networks within the lenses which acted as resonant circuits (e.g., with some combination of resistive, capacitive, and inductive components). In one embodiment a shift in system resonant frequency of 1.67 MHz was observed for a 1.5 mm radial expansion in a ring lens, demonstrating utility of wireless (i.e., no wired connection between the lens and the reading coil) frequency-based strain gauges for large-scale lens expansions with nanowire smart contact lenses. In some preliminary tests correspondence between pressure and frequency changes were not detected for lens expansion at test pressures of 0-49 mmHg. It is expected that design refinements will enable improved measurement sensitivity for small-scale contact lens flexing, as well as a correlation between resonant frequency shift and IOP changes.

Embodiments may include one or more of a contact lens containing a nanowire mesh network, an inductive reading coil to couple with the nanowire mesh network, and a portable signal detection module to obtain frequency measurements. In a system for demonstrating and testing embodiments, a Nanovna V2 portable Vector Network Analyzer (VNA) was used, due to its large bandwidth (50 kHz to 3 GHz), small size, and affordable price. The system measured frequency changes based on flexing/expansion of lenses with AgNW networks—the AgNW mesh has intrinsic inductive, capacitive, and resistive circuit characteristics which allow it to interact with the resonance frequency of the reading coil. As the lens expands with the eye (due to increasing IOP), these parameters change with the expansion of the mesh, shifting the resonance peak of the reading coil and mesh system. A correlation between resonance frequency and IOP may then be determined through experimental testing and calibration.

Several designs for both lens structure and circuit configuration were considered. The designs were a full (closed) lens with a solid circle of AgNW in the centre (FIG. 1A), a ring-shaped (open) lens with a ring of AgNW (FIG. 1B), a full lens with AgNW throughout (FIG. 1C), and a ring-shaped lens with a hole of 3-6 mm diameter in the centre, with embedded AgNW throughout (FIG. 1D). The lenses were between 6-14 mm in diameter. Other designs and configurations may of course be implemented.

The full lens with AgNW is possible because AgNW meshes, when deposited in very thin layers, can be transparent, providing minimal impact on the vision of the wearer [6]-[8]. The circuit design is very simple for this configuration relying on the various concentrations of the AgNW as well as the central hole size when a hole is added, and lends itself well to quick and reliable manufacturing, as the AgNW solution is easy to deposit without overflow.

The ring lenses demonstrated very high sensitivity, as the strain is concentrated around the edges of the lens where the nanowire mesh is located. One embodiment based on FIG. 1B where an inner diameter for the nanowire ring was about 5 mm and width was about 1 mm, and the hole diameter was about 4 mm, provided minimal impact on the vision of the wearer. Nanowire meshes may be made thicker and/or wider, e.g., for higher resistivity or sensitivity, as the opacity of the mesh is not a concern. However, this configuration is more difficult to manufacture, as the channel is narrow and prone to overflow when the nanowire solution is deposited.

To obtain the frequency readings, an inductive reading coil was connected to the VNA through the S11 port. The coil was placed 0.7 cm from the lens. The VNA performed sweeps of radiofrequency (RF) energy across a designated frequency range every second, with 101 sample points for each sweep.

Embodiments are further described by way of the following non-limiting examples.

### Examples

### 1. PDMS Lens Fabrication

PDMS was prepared using a ratio of 10:1 of base to curing agent—this ratio provided a good balance between elasticity of the lens and ability of the lens to adhere to the lubricated eye [2], [9]. To remove air bubbles from the PDMS, which can affect the structural integrity of the lens, the mixture was degassed for a minimum of 10 minutes under vacuum after being thoroughly mixed.

To form the lenses, the PDMS was poured into an aluminum mould. In one embodiment for making a lens as in FIG. 1B, the mould had a raised circular or ring-shaped channel to create a bottom layer for the contact lens with a channel in which to deposit the nanowires. The PDMS was then cured in an oven at 150 degrees Fahrenheit for 120 minutes. Before deposition, the channel in the bottom layer was treated using a corona surface treater to make the surface hydrophilic, allowing the nanowires to spread evenly when deposited. A smooth, thin (approx. 100 μm) top layer was then formed in a separate mould and bonded to the bottom layer after the nanowires had been deposited in the channel, forming an AgNW “sandwich”. Alternative methods of lens fabrication are described below.

2. Planar Lenses with 3D Printed Moulds

With thin layers of PDMS, planar sheets may be heated and moulded into a lens shape using a smooth aluminum mould. This technique was applied to the ring lens configuration, using two layers with the nanowire mesh embedded within. 3D printed planar moulds were created using an ANYCUBIC Photon Mono 3D printer-one mould for the bottom layer with the AgNW channel, with a channel depth of 100 μm and a total thickness of 150 μm, and another mould for the flat top sheet with a thickness of 100 μm. The AgNW channel had a width of 1 mm, and an inner diameter of 5 mm. The moulds were square shaped with exterior walls, forming a well for the PDMS. These walls determined the thickness of the PDMS layer. The moulds were made from ANYCUBIC Standard UV-Curing resin, with a curing time of 4 s per layer to ensure mould stiffness.

The moulds were first treated with two coatings of a spray-on mould releaser safe for use with resin moulds and left to dry for five minutes. The PDMS was then poured into the moulds after being thoroughly mixed and degassed. Using a flat edge, excess PDMS was scraped off the top of the moulds. The PDMS was then degassed again in the moulds to remove any air bubbles introduced in the pouring step. The moulds were then cured in an oven.

Problems were encountered with this process so an alternative method for making a smooth planar PDMS sheet was used instead. PDMS was prepared and deposited on a plastic slide and spun using a vacuum spin-coater. With this spin-coating method, it was difficult to reproduce layers of equal thickness over multiple trials. It was determined experimentally that, when one gram of PDMS mixture was placed directly in the centre of the slide and spun at 400 RPM for 45 seconds, sheets of PDMS with thicknesses ranging from 75 μm to 125 μm could be reliably reproduced.

To create the lens, the bottom sheet was surface treated for 30 seconds to make the surface hydrophilic. A total of 100 μl of nanowire solution then was deposited in four layers of 25 μl, with a rest period of 10 minutes in between each layer to ensure that the ethanol from the solution had completely evaporated. This multi-layer process ensures even coating of nanowires, and strong electrical connections [2], [10], [11]. For use with the bottom sheet, a section of 85 μm thick PDMS was cut and placed overtop of the AgNW mesh. The sheets were bonded using two droplets of PDMS spread across the surfaces and cured for 20 minutes at 150 degrees Fahrenheit. The completed lens had a thickness of 242 μm. The lens was then trimmed by hand into a circle with a diameter of 13 mm, and a 3 mm hole was then punched in the center of the lens.

The lens was then placed into a smooth lens-shaped aluminum mould, with an aluminum dome on top to press the lens into the mould. The shaped lens was then heated for 30 minutes at 150 degrees Fahrenheit and cooled for a subsequent hour in the mould. The resulting lens had a dome-like shape but contained several wrinkles, and visible cracks in the nanowire mesh rendering it unusable.

### 3. Curved Smooth Lenses

Another lens fabrication method utilized existing smooth aluminum moulds to create lenses without features. Lens-shaped aluminum moulds were coated with mould releaser, and PDMS was poured into the moulds after being degassed. When bubbles were present, the PDMS was degassed again, and then the top section of the mould was placed on top to form the lens. The lens was cured for 60 minutes at 150 degrees Fahrenheit and left to cool for another 20 minutes before separation. Once removed from the mould, the inside of the lens was treated with a corona surface treater. 100 μl of AgNW solution was then dropped into the centre of the treated lens in successive layers to form a mesh with a similar shape to a circle.

With this volume of AgNW solution, the resulting mesh was not transparent. A highly transparent mesh was obtained by dropping the minimum possible amount of 20 μl with the available laboratory equipment, however the mesh was extremely fragile and prone to cracking/flaking when curing. For testing proof-of-concept purposes, the thicker mesh was expected to be more useful. With this deposition technique, the majority of the nanowires were concentrated in the centre of the lens. The resulting mesh had a diameter of 8.0±0.5 mm. A second identical PDMS lens was created, without the nanowire mesh. This lens was then used to sandwich the AgNW lens, bonded with droplets of PDMS.

The finished lens had a total thickness of 470 μm. The thickness of the lenses is a significant drawback to this fabrication method, as the available moulds produced lenses sections around 230 μm thick. Furthermore, the AgNW mesh had a non-uniform nanowire distribution. For the purposes of proof of concept, however, this fabrication method proved useful.

### 4. Curved Ring Lens

A curved ring lens was made using a similar fabrication process. The top portion of the lens was created and nanowires were deposited to form a quasi-circular mesh. A 3 mm hole was then punched in the center of the lens to form an open ring, bordered with a ring-shaped nanowire mesh.

The AgNW mesh was prone to flaking if touched or left unprotected with a bottom layer of PDMS. Imperfections were spot-treated with a total of 50 μl of AgNW solution to complete the ring. To form the bottom layer, a smooth, curved PDMS lens was fabricated with a flat section of PDMS extending radially from the edge of the lens. The bottom lens half served as a rudimentary eye replica for testing purposes (henceforth referred to as the “PDMS eye”). The ring lens was then placed atop the PDMS eye and bonded with a thin layer of PDMS. The total thickness of this configuration was 438 μm.

### 5. Hydrogel Lens Fabrication

A hydrogel (pHEMA) lens was prepared using a ratio of 100 parts HEMA, 5.0 parts ethylene glycol dimethylacrylate (EGDMA), and 0.5 parts IGACURE 1173. To remove any impurities, all reagents were filtered through an inhibitor remover and wool cotton, then stored in an amber-colored glass vial.

A silver nanowire (AgNW) solution (0.5% in isopropyl alcohol or 5 mg/mL) was poured into a petri dish and left for twenty-four (24) hours to allow the isopropyl alcohol residue to evaporate. The AgNW were then weighed and mixed with the pHEMA solution at concentrations of 0.05%, 0.1%, and 0.2%.

To form the hydrogel lenses, the pHEMA mixture was poured into a polypropylene mold and cured using a UV curing machine (Omnicure S2000 Elite) for 10 minutes. After curing, the lenses were soaked in PBS solution for twenty-four (24) hours. The finished lens had a total thickness of about 250 μm. With this technique, the nanowires were dispersed substantially throughout the lenses (e.g., FIGS. 2A-2C for the three concentrations). Embodiments may be implemented as full (closed) lenses, or as ring-shaped (open) lenses have central holes with different sizes to provide a desired level of sensitivity, or in other configurations.

### 6. Reading Coil

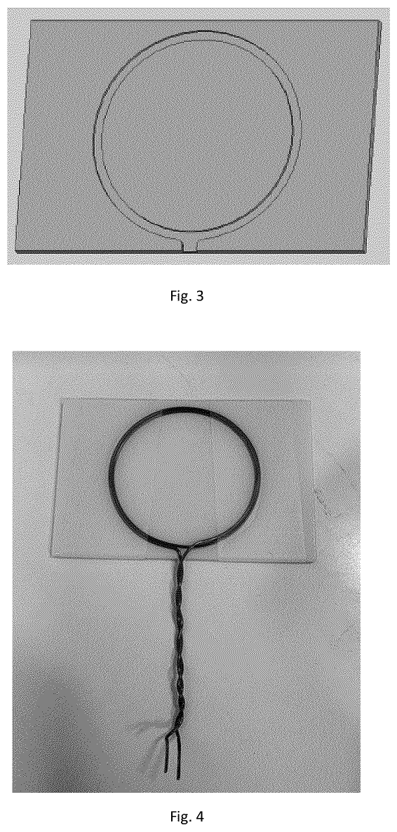

To make the measurement portion of the apparatus, an inductive reading coil was made to pair with the VNA. A planar coil holder (FIG. 3) was used. The coil wires were placed within the groove of the coil holder for two loops, the innermost having a diameter of 33.5 mm and the outermost having a diameter of 34.8 mm. The inductance of the coil was measured to be 740±50 μH. The coil was made using enamel-coated 22 AWG copper wire. The enamel coating was necessary as it protected against short circuit between the wires. The wires were held in place with clear tape. The tails from the coil were twisted to reduce electromagnetic interference with the main body of the coil. The ends of the wires had their enamel burnt off to allow for an electrical connection to be formed. One embodiment of a reading coil is shown in the photograph of FIG. 4.

### 7. VNA Setup

Prior to being used for measurements, an SMA cable was connected to the S11 port of the VNA. The VNA was calibrated in the Open, Load, and Short conditions using the calibration instruments provided with the VNA kit at the end of the SMA cable. The first calibration was performed over the entire range of the VNA—this calibration was not sufficient for accurate measurements but was used to provide a close approximation of the IOP measurement system resonance frequency when used for measurement. Once the approximate frequency was determined, a localized calibration was performed to increase measurement accuracy.

### 8. Lens Testing

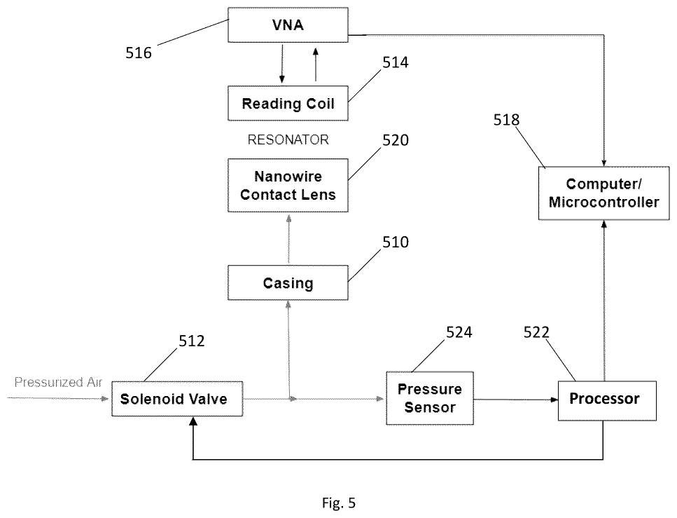

An experimental set up used to obtain frequency response data of the lenses is shown in FIG. 5. Referring to FIG. 5, a custom testing apparatus 510 was made to perform frequency response measurements with the different lenses for stable and known pressures. A base portion of the apparatus 510 housed a hollow chamber in the centre, with a small hole extending from the chamber through the side of the apparatus. A lid was also made with a 14 mm diameter hole in the centre, to sandwich the fringes of the PDMS eye forming an airtight seal, while allowing the centre portion of the eye to expand freely.

A small hose connected to an air supply through a solenoid 512 was placed into the hole in the side of the base. This allowed the system to be pressurized when a PDMS eye was placed between the lid and the base, and secured with tape. The lid had raised platforms to hold the reading coil 514 at a distance of 0.7 mm from the lens 520 on the PDMS eye. This was an approximation of the average distance a pair of glasses lens would sit from the wearer's eye, based on several measurements taken from individuals' glasses, where an implementation may include disposing the reading coil on a frame or peripheral portion of a lens of a pair of glasses. Thus the setup modelled a scenario in which the reading coil could be attached to or embedded in a pair of glasses to be worn by a subject to maintain the reading coil at the proper distance from a lens on the subject's eye for continuous IOP measurement throughout the day.

A VNA 516 was connected to a laptop computer 518, and frequency data was read and saved in real-time using the Nanovna-saver Python SDK [12]. A Python script was implemented to read and save the maximum gain and corresponding frequency for each sweep performed by the VNA.

To control the pressure, a processor 522 (e.g., Arduino Uno) was used to read pressure values with a Honeywell SSCDRRN001PDAA5 differential pressure sensor 524. The sensor had a range of ±55 mmHg around atmospheric pressure. Pressure values were read once every second, corresponding with the sweep timing from the VNA. The processor 522 was also used to open and close the solenoid 512 connected to the main pressure line at regular intervals, pressurizing and depressurizing the test apparatus.

When the lenses were combined with the testing apparatus, the full-mesh lens was paired with a plain PDMS eye for testing. The completed lens was unable to adhere properly to the PDMS eye during testing and it was not possible to obtain a complete set of data. All subsequent tests were performed using the ring-shaped lens due to reliability and time constraints.

### Results

### Transparency

Contact lenses require high transparency. To quantify the loss of transparency incurred by including nanowires, the change in the coefficient of optical transmission was measured. For this analysis, ASTM F1316, a test for measuring the transmissivity of transparent materials, was used. An Adafruit TSL2591 High Dynamic Range Digital Light Sensor was used as the photometer. It was placed in a box custom built to shield outside light. A lamp was placed approximately 40 cm above the photometer. For testing, sheets of pHEMA lens material were put above the photometer box, so that any light reaching the photometer was required to travel through the film. Tests were done in a room with the lights off and the windows shaded to control for ambient light conditions. During the test, four measurements were made. First, the lamp was turned on and the photometer was used to measure the intensity of the light in lux, represented by the variable Ls. Next, a pHEMA sheet was placed on top of the box and the photometer was used to measure the light intensity of the lamp measured through the film, Lst. ASTM F1316 recommends then placing a piece of black material over the light sources and repeating the two measurements. For this, a piece of black sandpaper was used. It was cut in a circular shape matching the diameter of the lamp shade. The measurement of luminance of the black surface through the transparency is represented by Lbt and the luminance of the black surface measured directly is Lb. Using these four values, the transmission coefficient T was determined by the following equation:

T=(Lst−Lbt)/(Ls−Lb)

where T is the optical transmittance of the sample expressed as a percentage in Table 1. For each measurement, L, 15 seconds of data was collected and the mean over that interval was used in the calculation of t.

TABLE 1
Transparency of pHEMA samples with various concentrations of nanowires.

| | | Thickness |
|---|---|---|
| Samples | Transperancy (%) | (mm) |
| 0.05% AgNW | 80.76 | 0.63 |
| 0.1% AgNW | 75.74 | 0.48-0.57 |
| 0.2% AgNW | 65.54 | 0.49 |
| pHEMA-Control | 92.87 | 0.82 |

### Lens-Coil Interaction

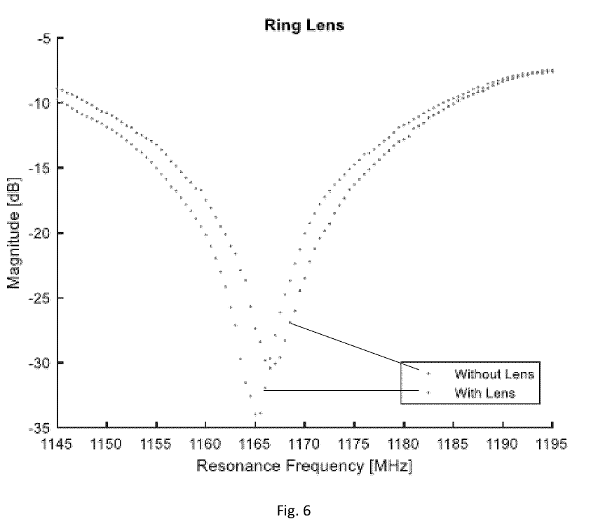

Seven measurements were taken of the resonance frequency of the reading coil. Readings were taken a minimum of two minutes after the circuit had been assembled to let vibrations, a significant source of noise, dissipate and acquire a stable reading. The ring lens was then placed within the testing apparatus, 0.7 cm away from the coil. Seven readings were taken with the lens included. This test served as a basic proof of concept (POC) to show that the introduction of the nanowire mesh circuit into the system changes the resonance frequency by a significant amount. As shown in FIG. 6, averaging over the seven trials, a 3 MHz shift in resonance frequency was measured when a ring lens was introduced. The resonance peak sharpened significantly, with an increase of 4.5 dB when the lens was introduced (see FIG. 6). This result shows a clear coupling interaction between the reading coil and the AgNW mesh circuit at a distance of 0.7 cm.

### Frequency Response

To determine the relationship between resonance frequency and pressure applied to the lens, several trial runs were performed using a ring lens and PDMS eye setup. The PDMS eye with a ring lens attached was placed between the lid and base of the testing apparatus and secured firmly with tape. Using the processor, pressure was applied and turned off at five second intervals. The pressure was varied between 0 and 49 mmHg. Clear expansion of the lens was observed visually with the applied pressure compared to atmospheric. The maximum measured expansion of the lens diameter, using a video camera with reference material, was approximately 0.3 mm. A significant change in resonance frequency was not detected, averaging over six trials. This is attributed to fluctuations in resonance frequency of up to 2 MHz observed during testing, due to environmental noise factors and poor electrical connections.

The pressure range used was beyond the maximum pressure commonly seen in glaucoma cases. For similar experiments, a maximum IOP of 40 mmHg was considered. This result suggests that there are limitations with the physical design of the lens and/or the sensitivity of the testing apparatus. It was noticed that a significant portion of the expansion occurred around the edge where the lid secured the PDMS eye to the base—the eye would stretch upward from the edge, rather than inflating uniformly, similar to a balloon. This non-uniform expansion likely reduced the radial expansion of the lens significantly and contributed to the lack of data.

### Manual Lens Deformation

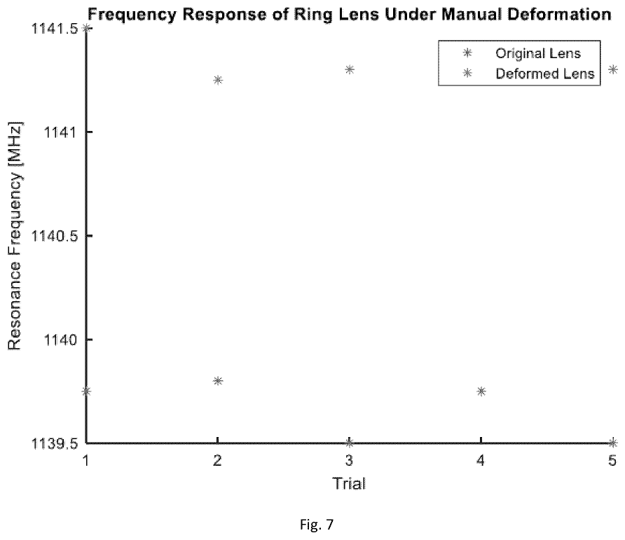

A further test to investigate whether lens deformation could be detected by the VNA and reading coil apparatus was performed by manually deforming the lens. Five measurements of the frequency response were made with the lens at equilibrium. The lens was then deformed by approximately 1.5 mm in diameter and placed beneath the reading coil. Five measurements were taken and are shown in the FIG. 7 (upper points, deformed lens; lower points, equilibrium (original)).

A clear increase in resonance frequency of approximately 1.67 MHz was measured compared to the lens at equilibrium. Although the amount of expansion and strain experienced by the lens are beyond that which would be achieved on a human eye with glaucoma, the result shows that even with a rudimentary ring lens and basic VNA, wireless strain measurements can be obtained according to the methods and apparatus described herein, and they may be adapted and optimized to measure IOP.

### Limitations and Sources of Error

The early stage of the fabrication process according to certain embodiments may have had a negative impact on the prototypes tested, but embodiments nevertheless clearly demonstrate the functionality and utility of lenses with embedded nanowire networks for IOP measuring and monitoring. The moulds used to create the closed lenses resulted in a lens thicker than intended, with a total center thickness of up to 500 μm. However, use of hydrogel as described herein allows thinner lenses to be made, which may address this issue. The 3D printing method showed some promise as an easily reproduceable and cost-effective method for creating custom moulds, provided that a high-heat resin could be used, and a smooth curved mould be created. Further refinements include improving the uniformity of nanowire distribution over a specific region of the lens (e.g., in the embodiments with ring structures) or across the entire lens (in embodiments with nanowire network throughout). For example, a more uniform nanowire channel around the edge of the ring lens would result in a significant increase in sensitivity, which would produce better results.

Other limitations of the set up were the electrical connections and the PDMS eye testing apparatus. A significant source of noise in the measured signal resulted from poor connections between the SMA adapter and the breadboard circuitry. Improved connections will reduce electrical noise and produce better results. During testing, tape was used to secure the lid of the testing apparatus to the base and create a seal with the PDMS eye. A more reliable way of securing the lid to the base of the testing apparatus, such as a clamp, would create an airtight uniform seal, leading to more even expansion and reduction of air leakage, and would also produce better results.

### Implementation

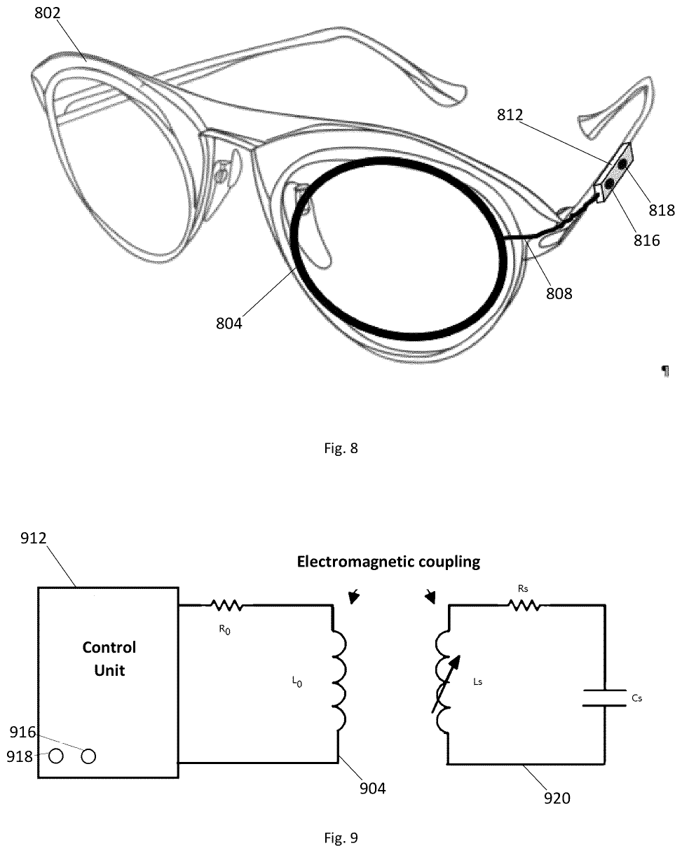

An example of an implementation suitable for ease of use in measuring and long-term monitoring of IOP is shown in FIG. 8. According to this embodiment, a reading coil 804 is disposed in or on frame or lens of a pair of glasses 802. The glasses 802 may be “dummy” glasses in that they have no actual lenses, or the lenses have no corrective optical properties. The reading coil 804 is connected 808 to a control unit 812 which may be mounted on or integrated or embedded in the arm of the glasses or elsewhere on the frame. The control unit 812 may include signal processing components and other components for use with the reading coil 804, such as, but not limited to, signal generator, oscillator, amplifier, analogue to digital converter (ADC), logic circuitry (i.e., processor), memory device, wireless communications device (e.g., Blue Tooth™), power supply (e.g, battery), etc., and push buttons 816, 818 or similar actuators for user input to control basic functions (e.g., power on/off, initiate or stop data acquisition and/or processing, communications pairing with receiving device, calibration (to a baseline signal, etc.)).

The control unit may be implemented in whole or in part using discrete (e.g., analogue) components and/or using digital technology, such as an integrated circuit (IC) implementation, which greatly reduces component cost and design complexity. Examples of suitable digital technologies include processors such as, but not limited to, digital signal processor (DSP), field programmable gate array (FPGA), application specific integrated circuit (ASIC), and microcontroller unit (MCU). One or more components of a control unit may be implemented in an algorithm using a suitable computer or hardware language (i.e., code) such as, for example, very high speed integrated circuit (VHSIC) hardware descriptive language (VHDL), register transfer language (RTL), or Verilog. Such an algorithm may be stored in a non-volatile memory of the controller and executed in, for example, a DSP, FPGA, ASIC, or MCU device of the controller. The control unit produces a signal at a selected frequency (e.g., an RF signal) that is output to the reading coil 804. Electromagnetic coupling is established between the reading coil 804 and an IOP measuring nanowire contact lens according to embodiments described herein. The reading coil exhibits a change in resonant frequency when the nanowire lens deforms, i.e., changes shape in response to a change in IOP, wherein the resonant frequency is related to IOP. The resonant frequency of the reading coil is measured and processed by the control unit 812, and data are sent to a connected device such as, for example, a smart phone, tablet, laptop computer, etc. over a wireless communications network. The connected device may then provide data processing, storage, analysis, and communication to a base station or server located, for example, at a clinic or doctor's office.

An implementation is shown schematically in the embodiment of FIG. 9. Referring to FIG. 9, a control unit 912 with actuators 916, 918 is connected to a reading coil 904, represented with an inductance Lo and resistance Ro. Magnetic coupling is established between the reading coil 904 and a nanowire lens 920 according to embodiments described herein, represented with a variable inductance Ls, resistance Rs, and capacitance Cs. The variable inductance Ls is achieved by deformation of the nanowire network in the nanowire lens on a subject's eye, the deformation resulting from a change in IOP. As a result of the deformation, a resonant frequency of the reading coil 904 changes, which is detect and processed by the control unit 912.

All cited publications are incorporated herein by reference in their entirety.

### EQUIVALENTS

It will be appreciated that modifications may be made to the embodiments described herein without departing from the scope of the invention. Accordingly, the invention should not be limited by the specific embodiments set forth, but should be given the broadest interpretation consistent with the teachings of the description as a whole.

### REFERENCES

[1] “About Glaucoma,” Glaucoma Research Society of Canada. https://www.glaucomaresearch.ca/about/about-glaucoma/(accessed Oct. 20, 2022).
[2] R. Helgason and Y. Lai, “Increased sensitivity of smart contact lenses for continuous intraocular pressure measurement using ring-shaped design,” Flex. Print. Electron., vol. 7, no. 2, p. 024005, June 2022, doi: 10.1088/2058-8585/ac73ca.
[3] “Glaucoma|National Eye Institute.” https://www.nei.nih.gov/learn-about-eye-health/eye-conditions-and-diseases/glaucoma (accessed Oct. 18, 2022).
[4] H. An et al., “High-sensitivity liquid-metal-based contact lens sensor for continuous intraocular pressure monitoring,” J. Micromech. Microeng., vol. 31, no. 3, p. 035006, February 2021, doi: 10.1088/1361-6439/abd8e0.
[5] M. Lira, C. Pereira, M. E. C. D. Real Oliveira, and E. M. S. Castanheira, “Importance of contact lens power and thickness in oxygen transmissibility,” Cont Lens Anterior Eye, vol. 38, no. 2, pp. 120-126, April 2015, doi: 10.1016/j.clae.2014.12.002.
[6] D. Kumar, V. Stoichkov, E. Brousseau, G. C. Smith, and J. Kettle, “High performing AgNW transparent conducting electrodes with a sheet resistance of 2.5Ω Sq-1 based upon a roll-to-roll compatible post-processing technique,” Nanoscale, vol. 11, no. 12, pp. 5760-5769 March 2019, doi: 10.1039/C8NR07974A.
[7] R. E. Triambulo, H.-G. Cheong, and J.-W. Park, “All-solution-processed foldable transparent electrodes of Ag nanowire mesh and metal matrix films for flexible electronics,” Organic Electronics, vol. 15, no. 11, pp. 2685-2695 November 2014, doi: 10.1016/j.orgel.2014.07.039.
[8] G. Khanarian et al., “The optical and electrical properties of silver nanowire mesh films,” Journal of Applied Physics, vol. 114, no. 2, p. 024302, July 2013, doi: 10.1063/1.4812390.
[9] S. Vlassov et al., “Adhesion and Mechanical Properties of PDMS-Based Materials Probed with AFM: A Review,” REVIEWS ON ADVANCED MATERIALS SCIENCE, vol. 56, no. 1, pp. 62-78, May 2018, doi: 10.1515/rams-2018-0038.
[10] M. Amjadi, A. Pichitpajongkit, S. Lee, S. Ryu, and I. Park, “Highly Stretchable and Sensitive Strain Sensor Based on Silver Nanowire-Elastomer Nanocomposite,” ACS Nano, vol. 8, no. 5, pp. 5154-5163 May 2014, doi: 10.1021/nn501204t.
[11] J. Y. Song, J. H. Oh, D. Choi, and S. M. Park, “Highly efficient patterning technique for silver nanowire electrodes by electrospray deposition and its application to self-powered triboelectric tactile sensor,” Sci Rep, vol. 11, no. 1, p. 21437, December 2021, doi: 10.1038/s41598-021-01043-6.
[12] “NanoVNASaver.” NanoVNA-Saver, Nov. 27, 2022. Accessed: Nov. 29, 2022. [Online]. Available: https://github.com/NanoVNA-Saver/nanovna-saver

1. A contact lens comprising:
a polymer matrix; and
a nanowire network embedded in the polymer matrix.

2. Apparatus for measuring intraocular pressure (IOP), comprising:
the contact lens of claim 1; and
an electrically conductive coil adapted to share radiofrequency (RF) energy with the nanowire network of the contact lens.

3. The apparatus of claim 2, wherein the electrically conductive coil has a selected resonant frequency.

4. The apparatus of claim 3, wherein the resonant frequency changes in response to a change in configuration of the nanowire network.

5. The apparatus of claim 4, wherein the change in resonant frequency is correlated with a change in IOP.

6. The apparatus of claim 2, further comprising a device connected to the electrically conductive coil that produces and measures the frequency of the RF energy.

7. The apparatus of claim 2, wherein the electrically conductive coil is connected to a control unit;
wherein the control unit comprises one or more of signal generator, oscillator, amplifier, analogue to digital converter (ADC), logic circuitry, processor, memory device, wireless communications device, power supply, and actuator.

8. The apparatus of claim 7, wherein the control unit provides one or more of data acquisition, processing, identification of resonant frequency, and wireless data communications to a connected device.

9. The apparatus of claim 7, wherein the electrically conductive coil and the control unit are adapted to be worn by a user.

10. The apparatus of claim 7, wherein the electrically conductive coil and the control unit are adapted to be mounted on a pair of glasses.

11. The apparatus of claim 1, wherein the polymer matrix comprises at least one of a hydrogel and polydimethylsiloxane.

12. The apparatus of claim 1, wherein the polymer matrix comprises poly (2-hydroxyethyl methacrylate).

13. A method for measuring and/or monitoring IOP of a subject, comprising:
disposing a contact lens on the subject's eye, the contact lens comprising a polymer matrix and a nanowire network embedded in the polymer matrix;
disposing an electrically conductive coil in close proximity to the contact lens, wherein the electrically conductive coil electromagnetically couples with the nanowire network of the contact lens;
measuring a resonant frequency of the electrically conductive coil; and
correlating the resonant frequency with the IOP of the subject.

14. The method of claim 13, comprising measuring the resonant frequency of the electrically conductive coil at two or more instances in time; and
correlating a change in resonant frequency over the two or more instances in time with a change in IOP of the subject.

15. The method of claim 13, comprising connecting the electrically conductive coil to a control unit;
wherein the control unit comprises one or more of signal generator, oscillator, amplifier, analogue to digital converter (ADC), logic circuitry, processor, memory device, wireless communications device, power supply, and actuator.

16. The method of claim 15, wherein the control unit provides one or more of data acquisition, processing, identification of resonant frequency, and wireless data communications to a connected device.

17. The method of claim 15, wherein the electrically conductive coil and the control unit are adapted to be worn by a user.

18. The method of claim 15, wherein the electrically conductive coil and the control unit are adapted to be mounted on a pair of glasses.

19. The method of claim 13, wherein the polymer matrix comprises at least one of a hydrogel and polydimethylsiloxane.

20. The method of claim 13, wherein the polymer matrix comprises poly (2-hydroxyethyl methacrylate).
